(19) United States
(12) Patent Application Publication
(10) Pub. No.: US 2016/0296574 A1
(43) Pub. Date: Oct. 13, 2016

(54) EPHEDRA ALATA EXTRACTS AND METHODS OF USE THEREOF

(71) Applicants: Osama Mansour Murad, Amman (JO); Yousef Al-Abed, Dix Hills, NY (US)

(72) Inventors: Osama Mansour Murad, Amman (JO); Yousef Al-Abed, Dix Hills, NY (US)

(21) Appl. No.: 15/036,470

(22) PCT Filed: Nov. 17, 2014
(86) PCT No.: PCT/US14/65925

§ 371 Date: May 13, 2016

Related U.S. Application Data
(60) Provisional application No. 61/904,762, filed on Nov. 15, 2013.

(51) Int. Cl. A61K 36/17; A61K 31/34; A61K 9/00; A61K 31/7032; A61K 9/19

(52) U.S. Cl. CPC A61K 36/17; A61K 31/7032; A61K 9/19; A61K 9/0053; A61K 31/34; A61K 2236/331; A61K 2236/37; A61K 2236/53; A61K 2236/51

(57) ABSTRACT

Ephedra alata aqueous extracts, powders and constituents thereof are encompassed herein, as are compositions thereof. Methods of using same for treating inflammatory diseases, autoimmune diseases, cancers, viral diseases, and neurodegenerative diseases are also envisioned.

### CROSS REFERENCE TO RELATED APPLICATION

This application claims priority under 35 USC §119(e) from U.S. Provisional Application Ser. No. 61/904,672, filed Nov. 15, 2013, which application is herein specifically incorporated by reference in its entirety.

### FIELD OF THE INVENTION

The present invention pertains generally to the use of herbal extracts of Ephedra alata, active ingredients, and various combinations thereof for boosting or stimulating immune responses in humans and animals and for treating humans and animals suffering from inflammatory diseases, autoimmune diseases, cancer, viral infections, and/or neurodegenerative diseases.

### BACKGROUND OF THE INVENTION

The citation of references herein shall not be construed as an admission that such is prior art to the present invention.

The efficacy of various herbal remedies, extracts, potions and treatments is well established, and therapeutic herbal products are increasingly recognized as valuable, particularly under circumstances wherein access to standard care medication is limited or doesn't exist or wherein administration of standard care medication has proven ineffectual.

### SUMMARY OF THE INVENTION

The present inventors have made the surprising discovery that aqueous extracts of Ephedra alata exhibit therapeutic properties that have been shown to be efficacious for treating a variety of diseases and conditions in human subjects. Accordingly, the present inventors have purified and identified aqueous extracts of Ephedra alata that may be used to advantage for treating subjects afflicted with such diseases and conditions. Aqueous extracts of Ephedra alata for use in treating these diseases and in the preparation of medicaments for the treatment of same are also envisioned. Also encompassed herein are similar methods, uses, and medicaments whereby Ephedra alata powder or active constituents of Ephedra alata aqueous extracts or powders are implemented.

The present inventors have, furthermore, corroborated and extended the promising results observed in humans in animal models of human disease. More particularly, the present inventors have, for example, demonstrated that administration of Epehdra alata aqueous extracts to a mouse strain that represents an animal model of multiple sclerosis and ataxia confers significant therapeutic benefit to the treated animals.

In a particular aspect, an aqueous extract or powder or constituent thereof generated from Ephedra alata is described. Accordingly, the present invention encompasses an aqueous extract or powder or constituent thereof purified from Ephedra alata and/or constituents thereof and compositions of these aqueous extracts, powders and/or constituents thereof. Such aqueous extracts or powders are enriched with respect to constituents (e.g., active ingredients) of the Ephedra alata plant.

In a particular embodiment, the aqueous extract or powder or constituent thereof comprises at least (but not limited to) one of p-coumaric, ephedralone, furanofuran lignan, syringaresinol, nilocitin and digallooylglucose or derivatives thereof. The aqueous extract could include other compounds that are not yet characterized.

In another particular embodiment, the aqueous extract or powder comprises at least one of alkaloidal, lignan and phenolic constituents of Ephedra alata.

Also encompassed herein is a composition comprising the Ephedra alata aqueous extract or powder or a constituent thereof and a pharmaceutically acceptable carrier.

Compositions comprising the aqueous extract or powder or a constituent thereof in combination with at least one of p-coumaric, ephedralone, furanofuran lignan, syringaresinol, nilocitin and digallooylglucose or derivatives thereof and a pharmaceutically acceptable carrier are also envisioned. Also hereby encompassed are compositions comprising the aqueous extract or powder or a constituent thereof in combination with at least one of alkaloidal, lignan and phenolic constituents of Ephedra alata and a pharmaceutically acceptable carrier.

In a further aspect, a method for making the aqueous extract of Ephedra alata is presented, the method comprising: processing an Ephedra alata plant or specific parts thereof in an aqueous solution to generate a supernatant comprising solubilized agents (e.g., active constituents) and insoluble material and removing the insoluble material from the supernatant, thereby generating the Ephedra alata aqueous extract. Ephedra alata aqueous extracts so generated can optionally be concentrated or further enriched to increase the concentration of active constituents present therein.

In a particular embodiment, the processing comprises boiling Ephedra alata plant or specific parts thereof in water. The Ephedra alata plant or specific parts thereof may be fresh or dehydrated. In a more particular embodiment, the specific parts are leaves and small branches or leaves of the Ephedra alata plant.

In another particular embodiment, the removing comprises gravity separation, centrifugation, and/or sieving through a filter.

In yet another embodiment, the method further comprises reducing volume of the Ephedra alata aqueous extract to generate a concentrated or further enriched Ephedra alata aqueous extract. In a particular embodiment thereof, the reducing is achieved by lyophilization. The method may further comprise mixing the Ephedra alata aqueous extract or the concentrated Ephedra alata aqueous extract with a pharmaceutically acceptable excipient or carrier.

In a further aspect, a method for making an Ephedra alata powder is presented, the method comprising: processing an Ephedra alata plant or specific parts thereof to generate a dehydrated Ephedra alata plant product; and grinding the dehydrated Ephedra alata plant product to generate a ground mixture thereof, thereby generating the Ephedra alata powder. The Ephedra alata powder so generated is enriched with respect to constituents (e.g., active ingredients) of the Ephedra alata plant.

In a particular embodiment, the processing comprises washing and drying the Ephedra alata plant or specific parts thereof. In a more particular embodiment, the processing further comprises removal of large branches.

In another particular embodiment, the specific parts are leaves and small branches or leaves of the Ephedra alata plant.

In a further embodiment, the method further comprises sieving and regrinding the ground mixture.

In a still further embodiment, the method comprises mixing the Ephedra alata powder with a pharmaceutically acceptable excipient or carrier.

In another aspect, a method for treating a subject in need thereof with an Ephedra alata aqueous extract or powder or a constituent thereof described herein or a composition described herein is presented, the method comprising administering a therapeutically effective amount of the Ephedra alata aqueous extract or powder or a constituent thereof or the composition to the subject in need thereof.

In a particular embodiment of methods described herein, the subject is an animal (e.g., a mammal). In a more particular embodiment, the mammal is a human.

In another particular embodiment, the subject is afflicted with an inflammatory disease, an autoimmune disease, a cancer, a viral disease, or a neurodegenerative disease. Exemplary such diseases and conditions are listed in Tables 1 and 2. In a more particular embodiment thereof, the subject is afflicted with an inflammatory disease or an autoimmune disease. In a still more particular embodiment thereof, the subject is afflicted with multiple sclerosis or psoriasis.

In yet another particular embodiment, the Ephedra alata aqueous extract or powder, or constituent thereof or composition thereof is administered orally, topically, intratumorally, intranasally, intramuscularly, or intravenously. In a more particular embodiment, the Ephedra alata aqueous extract or powder or a constituent thereof or the composition is administered orally.

Also encompassed herein is a method for formulating the aqueous extract or powder of Ephedra alata described herein or a constituent thereof or a composition described herein into a capsule, a tablet, a topical lotion, or a nasal spray, or a solution suitable for intravenous, intramuscular, or intratumoral administration.

Also encompassed herein is the aqueous extract or powder of Ephedra alata described herein or a constituent thereof or a composition described herein for use in treating an inflammatory disease, an autoimmune disease, a cancer, a viral disease, or a neurodegenerative disease comprising administering the Ephedra alata aqueous extract or powder or a constituent thereof or the composition in a therapeutically effective amount to a subject afflicted with the inflammatory disease, autoimmune disease, cancer, viral disease, or neurodegenerative disease. Exemplary such diseases and conditions are listed in Table 1 and 2. In a more particular embodiment thereof, the disease is an inflammatory disease or an autoimmune disease. In a still more particular embodiment thereof, the disease is multiple sclerosis or psoriasis.

Also encompassed herein is the use of the Ephedra alata aqueous extract or powder described herein or a constituent thereof or a composition described herein in the preparation of a medicament for the treatment of an inflammatory disease, an autoimmune disease, a cancer, a viral disease, or a neurodegenerative disease comprising administering the aqueous extract or powder or constituent thereof or the composition in a therapeutically effective amount to a subject afflicted with the inflammatory disease, autoimmune disease, cancer, viral disease, or neurodegenerative disease. Exemplary such diseases and conditions are listed in Tables 1 and 2. In a more particular embodiment thereof, the disease is an inflammatory disease or an autoimmune disease. In a still more particular embodiment thereof, the disease is multiple sclerosis or psoriasis.

Other objects and advantages will become apparent to those skilled in the art from a review of the ensuing detailed description, which proceeds with reference to the attendant claims.

### BRIEF DESCRIPTION OF THE DRAWINGS

FIGS. 1A and B show graphs depicting the therapeutic benefit conferred by administration of Ephedra alata aqueous extract to female progressive/ataxia Experimental Autoimmune Encephalomyelitis (EAE) mice. A) presents the EAE score for treated and untreated females. B) reflects the degree of ataxia in treated and untreated females.

FIGS. 2A and B show graphs depicting the therapeutic benefit conferred by administration of Ephedra alata aqueous extract to male progressive/ataxia Experimental Autoimmune Encephalomyelitis (EAE) mice. A) presents the EAE score for treated and untreated males. B) reflects the degree of ataxia in treated and untreated males.

### DETAILED DESCRIPTION OF THE INVENTION

The present inventors have discovered that Ephedra alata, a shrub that grows in North Africa, southern Europe, the Americas, and Asia is a valuable source of therapeutic agents. It is noteworthy that Ephedra alata has not been previously identified by practitioners of folk or herbal medicine and few reports have investigated this plant. One study identified a few compounds of Ephedra alata in a chloroform extract derived therefrom, which compounds included alkaloidal, lignan and phenolic constituents [Nawwar et al. (1985) Phytochemistry 24, 878-879, the entire content of which is incorporated herein by reference]. It is significant that the extract described herein differs from that previously described for a variety of reasons, including the fact that it is generated from an aqueous extract and is compositionally distinct. The present findings describe the first biological studies performed using Ephedra alata extracts, p and/or constituents thereof and demonstrate efficacy of same for treating a variety of diseases/conditions in humans.

As indicated in the Examples presented herein, all human patients received Ephedra alata aqueous extract. More particularly, each patient received twice daily 50 ml of the aqueous extract (25 mg per dose), which was administered orally. After lyophilization, the concentration of the crude extract was 0.5 mg/ml. The duration of the treatment regimen was dependent on the kind of disease as well as the disease state (early or late state) with which the patient presented. Generally, the treatment length ranged from 1-24 months.

As shown, for example, in Tables 1-3, patients with a variety of inflammatory and autoimmune diseases responded favorably to treatment with Ephedra alata aqueous extract, which was administered and synthesized as described herein.

TABLE 1
List of inflammatory and autoimmune diseases

| | number | |
|---|---|---|
| Disease | of cases | improvement (%) |
| 1-multiple sclerosis | 175 | 70-95 |
| 2-Psoriasis | 18 | 100 |
| 3-Renal failure | 1 | 100 |
| 4-Vitiligo | 3 | 80 |
| 5-Breast cancer | 3 | 95 |
| 6-Liver cancer | 3 | 85 |
| 7-Lymphoma | 2 | 75 |
| 8-Type 1 diabetes | 4 | 80 |
| 9. Prostate hyperplasia & Prostate cancer) | 10 | 90 |
| 10. Bladder cancer | 1 | 100 |
| 11. Pancreatic cancer | 2 | 100 |
| 12. brain tumor | 1 | in progress |
| 13. Glioma | 1 | in progress |
| 14: ALS | 1 | in progress |
| 15: Asthma | 3 | 60-80 |
| 16: Colitis | 5 | 50-90 |
| 17: Crohn's disease | 3 | in progress |
| 18: Rehab | 4 | in progress |

(stroke patients; mobility in patients is improved following treatment)

| | | |
|---|---|---|
| 19: irritative colitis | 9 | 50-90 |

(neural origin)

| | | |
|---|---|---|
| 20: kidney failure | 4 | 80-90 |

(one of the four patients suffers from kidney function impairment
as a result of the general toxicity associated with Cisplatin*)

| | | |
|---|---|---|
| 21: hepatitis C | 2 | 60 |
| 22: enuresis | 3 | 100 |

(immediate action-patients responded within the first 24-48 h)
*It is noteworthy that the extract also reduced cisplatin-induced toxicity of the kidney in a mouse model of cisplatin toxicity tested.

TABLE 2
List of neurodegenerative diseases

| Disease | number of cases | improvement (%) |
|---|---|---|
| 1. AD | 3 | 80 |
| 2. PD | 3 | 95 |
| 3. Autism | 1 | in progress |
| 4. Huntington's | 2 | in progress |

Abbreviations:
Alzheimer's Disease (AD),
Parkinson's Disease (PD),
Huntington's Disease (Huntington's)

Explanations of Improvement

Multiple sclerosis: Improvement in these patients was observed/evaluated in the following aspects—control of urination, improved bowel movement control, improved physical ability, and potentially most significantly, a decrease in the size and number of the lesions in the brain and spinal cord as revealed by magnetic resonance imaging (MRI).

Psoriasis: Improvement was observed in the disappearance of skin scales and return of the skin to normal appearance.

Renal failure: Improvement was observed in the decrease of creatinine levels from around 7 to 0.9.

Vitiligo: Improvement was observed in the return of skin color to an almost natural color of the patient's skin.

Breast cancer: The size of lesions was reduced significantly to achieve almost complete regression.

Liver cancer: Patients were in stage 4 and the lesions in the liver disappeared completely from the liver and their normal life was restored for 12-18 months.

Lymphoma: Improvement was observed in increased white blood cell (WBC) counts and treated patients have resumed their normal lives without the need for isolation.

Type 1 diabetes: Treatment significantly reduced the use of insulin. Also a possible restoration of the islets was evident by the increased level of c-peptide values from about zero to 1.5.

Prostate hyperplasia and prostate cancer: Restoration of normal urine flow within 24 hours and a decrease in PSA readings to normal levels.

AD: Improvement was observed in these patients as evidenced by the restoration of their memory and return to their previous life style.

Neuromuscular disease: Restoration of movement and daily activities of one patient who was completely paralyzed.

Percent (%) improvement, as presented in Tables 1 and 2, was determined on an individual basis and reflects clinical improvement as evidenced by symptomatic relief and/or quantitative measurements of disease indicators. With regard to diabetes, for example, levels of c-peptide can be used as a disease indicator.

TABLE 3
Regimen of Treatments

| Disease | Duration | Endpoint |
|---|---|---|
| Multiple | 4-6 months for newly | patients reached a state of |
| sclerosis | diagnosed | satisfaction |
| Psoriasis | 2-4 months | skin is normal |
| Renal failure | 6 months | creatinine levels were normal |
| Vitiligo | 4-6 months | skin color is back to normal |
| Breast cancer | 4-12 months | lesion vanished and cancer |
| | | markers are not detectable |
| Liver cancer | 6-18 months | ultrasound images |
| | | and tests show the |
| | | lesions in the liver are cleared |
| Lymphoma | 4-6 months for newly | WBC counts are normal |
| | diagnosed | |
| Type 1 diabetes | 4-8 months | still on treatment, insulin |
| | | intake reduced by 90% |
| Prostate | 1-4 weeks | discontinued after four weeks |
| hyperplasia | | |
| Prostate cancer | 4-12 months | PSA readings and ratio |
| | | return to normal |
| AD | 12-18 months | still on treatment |
| PD | 4-6 months for newly | continuous treatment |
| | diagnosed cases | |

In light of the findings presented herein, Ephedra alata aqueous extract is efficacious for human patients suffering from a variety of maladies and clearly confers significant symptomatic relief and a reduction in detectable disease.

Definitions

The term “comprise” is generally used in the sense of include, that is to say permitting the presence of one or more features or components.

The term “consisting essentially of” is used herein to limit the invention to the specified materials or steps and those that do not materially affect the basic and novel characteristics of the claimed products, compositions, and methods, as understood from a reading of the present specification. With respect to a composition, for example, the phrase includes the composition per se and modifications or additions thereto that would not affect the basic and novel characteristics of the composition.

The term “isolated” refers to the state in which agents of the invention may be used in accordance with methods described herein. Isolated agents are free or substantially free of material with which they are naturally associated such as other agents, polypeptides or nucleic acids with which they are found in their natural environment, or the environment in which they are prepared (e.g. cell culture) when such preparation is by recombinant DNA technology practiced in vitro or in vivo. Extracts and agents described herein may be formulated with diluents or adjuvants and still for practical purposes be isolated—for example the agents will normally be mixed with gelatin or other carriers if used to coat microtiter plates for use in immunoassays, or will be mixed with pharmaceutically acceptable carriers or diluents when used in diagnosis or therapy.

As used herein, “pg” means picogram, “ng” means nanogram, “ug” or “μg” mean microgram, “mg” means milligram, “ul” or “μl” mean microliter, “ml” means milliliter, “l” or “L” means liter.

The term “therapeutically effective amount” refers to the amount of an extract, agent, or compound that, when administered to a subject for treating a disease, is sufficient to effect such treatment of the disease. The “therapeutically effective amount” can vary depending on the compound, the disease and its severity, and the age, weight, etc., of the subject to be treated.

The term “treating” or “treatment” of any disease or infection refers, in one embodiment, to ameliorating the disease or infection (i.e., arresting the disease or reducing the manifestation, extent or severity of at least one of the clinical symptoms thereof). In another embodiment ‘treating’ or ‘treatment’ refers to ameliorating at least one physical parameter, which may not be discernible by the subject. In yet another embodiment, ‘treating’ or ‘treatment’ refers to modulating the disease or infection, either physically, (e.g., stabilization of a discernible symptom), physiologically, (e.g., stabilization of a physical parameter), or both. In a further embodiment, ‘treating’ or ‘treatment’ relates to slowing the progression of a disease.

The phrase “pharmaceutically acceptable” refers to molecular entities and compositions that are physiologically tolerable and do not typically produce an allergic or similar untoward reaction, such as gastric upset, dizziness and the like, when administered to a human.

Extracts, agents, compounds, and compositions of the present invention may be administered to a patient in need of treatment via any suitable route, including by intravenous, intraperitoneal, intramuscular injection, or orally. The precise dose will depend upon a number of factors, including whether the extract, agent, compound, or composition is for treatment or for prevention. The dosage or dosing regimen of an adult patient may be proportionally adjusted for children and infants, and also adjusted for other administration or other formats, in proportion for example to molecular weight or immune response. Administration or treatments may be repeated at appropriate intervals, at the discretion of the physician.

Extracts, agents, compounds, and compositions described herein are generally administered in the form of a pharmaceutical composition, which may comprise at least one component in addition to the agents and compounds. Pharmaceutical compositions according to the present invention, and for use in accordance with the present invention, may comprise, in addition to active ingredient, a pharmaceutically acceptable excipient, carrier, buffer, stabilizer or other materials known to those skilled in the art. Such materials should be non-toxic and should not interfere with the efficacy of the active ingredient. The precise nature of the carrier or other material will depend on the route of administration, which may be oral, or by injection, e.g. intravenous, or by deposition at a tumor site. Pharmaceutically acceptable excipients, carriers, buffers, stabilizers or other materials may be natural or synthetic (i.e., manmade or synthesized). In an embodiment wherein the pharmaceutically acceptable excipient, carrier, buffer, stabilizer or other material is natural, it is purified from or derived from a source other than Ephedra alata.

Pharmaceutical compositions for oral administration may be in tablet, capsule, powder or liquid form. A tablet may comprise a solid carrier such as gelatin or an adjuvant. Liquid pharmaceutical compositions generally comprise a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included.

Capsules, for example, may be made by cleaning and drying the leaves and branches of Ephedra alata plants and grinding the clean, dried leaves and branches to a very fine powder having a consistency similar to that of flour. The pulverized, fine powder may be encapsulated in, for example, 00 gelatin capsules, which are available for purchase from a number of manufacturers. Approximately 500 mg of pulverized, fine powder of Ephedra alata is typically used to fill each 00 capsule.

More particularly, a method for making the pulverized, fine powder from Ephedra alata plants is as follows: the leaves and small branches are washed with water and dried until they are dehydrated and then large branches are removed. Only small branches to which leaves are attached are used in the preparation of the pulverized, fine powder of Ephedra alata. The leaves amount to approximately 5% of the total weight of the plant. Although not wishing to be bound by theory, the present inventors believe that the active constituents are found in the leaves of the Ephedra alata plant.

For the preparation of the capsules, the dehydrated leaves and branches are ground and the ground mixture is sieved using a large coarse sieve. After the step of sieving, the sieved ground mixture is ground again into a very fine powder with a consistency similar to that of flour (e.g., processed wheat flour used in making baked goods like bread). As indicated above, the very fine Ephedra alata powder is dispensed into 00 gelatin capsules weighing approximately 500 mg each.

The dosing regimen envisioned ranges from one capsule (˜500 mg) a day to 10 capsules (˜500 mg each) per day. The range may be optimized based on the opinion of a skilled practitioner depending on the disease and its severity. In a particular embodiment, one 500 mg capsule may be administered per day. In another particular embodiment, two 500 mg capsules may be administered per day (1 in the morning and 1 in the evening). In yet another particular embodiment, three 500 mg capsules may be administered per day (2 in the morning and 1 in the evening; 1 in the morning and 2 in the evening; or 1 three times per day; or the like). In a particular embodiment, four 500 mg capsules may be administered per day (2 in the morning and 2 in the evening).

Incorporation of the very fine Ephedra alata powder into, for example, capsules is thought to promote the effect of the active ingredients of the plant by taking advantage of digestive enzymes present in the gastrointestinal tract (particularly the acidic milieu of the stomach) to extract further the active constituents of the purified powder (i.e., the fine particles of the leaves) and thereby confer additional beneficial pharmacological effects due to release of the active constituents or compounds therein. The same reasoning applies equally to incorporation of the very fine Ephedra alata powder into other vehicles for oral administration such as, for example, tablets (coated or uncoated), sachets, or the like.

As used herein, the terms “Ephedra alata powder” or “ very fine Ephedra alata powder ” or “ pulverized, fine powder of Ephedra alata” may be used to refer to a manmade substance comprising fine particles made by grinding desiccated Ephedra alata plant parts (e.g., leaves) that comprise active constituents, agents or compounds of Ephedra alata. The active constituents, agents or compounds of Ephedra alata are present in the Ephedra alata powder at concentrated levels relative to their levels in the plant parts from which the powder is derived and purified. The active constituents, agents or compounds of Ephedra alata are also present in a highly particularized matrix of fine particles unlike any Ephedra alata plant part found in nature. The Ephedra alata powder is thus, a manmade substance having structural properties that differ from those of the Ephedra alata plant parts from which it is generated and purified and thus, can only be generated via human intervention.

In a particular embodiment, the Ephedra alata powder is incorporated into a composition that further comprises a pharmaceutically acceptable carrier or excipient. In a more particular embodiment thereof, the pharmaceutically acceptable carrier or excipient is a synthetic pharmaceutically acceptable carrier or excipient.

Water extracts of Ephedra alata are also envisioned herein. To this end, fresh or dried Ephedra alata plant is boiled in water to generate a water-based supernatant comprising active ingredients of the plant. It is to be understood that active ingredients extracted in water, which extraction process is enhanced and accelerated by application of heat, are present in the water-based supernatant at concentrations and in relative ratios that differ from extracts generated using other extraction methods (e.g., extraction solutions other than water such as, for example, organic solvents like methanolic and hexanic solutions).

In a particular embodiment, 140 g of fresh or dried Ephedra alata plant is boiled in 6 L of water to generate a water-based supernatant. The water-based supernatant can be used directly on an animal or a human or dried under, for example, lyophilization conditions. The dried material can be re-constituted in aqueous solution or other formulation for the purpose of in vitro and/or in vivo testing. In a particular embodiment, the extract concentration of the crude extract may be approximately 0.5 mg/ml after lyophilization.

In a particular embodiment of the preparation of the water extract, 140 grams of the dried leaves are added to 6 L of boiling water for one hour, after which the water extract is left at room temperature to cool and then filtered. The filtered Ephedra alata water extract (aqueous extract) is subsequently bottled and maintained in a refrigerator.

In one embodiment, the filtered Ephedra alata water extract comprises no preservatives. In another embodiment, the filtered Ephedra alata water extract is supplemented with a natural or a non-natural preservative to increase the shelf life of the extract.

The dosing regimen envisioned ranges from about one 50 ml dose to ten 50 ml doses of filtered Ephedra alata water extract (aqueous extract) per day. The range may be optimized based on the opinion of a skilled practitioner depending on the disease and its severity. In a particular embodiment, the filtered Ephedra alata water extract is administered to a subject in need thereof in doses of approximately 50 ml twice daily (total of 100 ml per day). In another particular embodiment, the filtered Ephedra alata water extract is administered to a subject in need thereof in two doses of approximately 100 ml twice daily (total of 200 ml per day).

As used herein, the terms “Ephedra alata aqueous extract” or “Ephedra alata water extract” refer to a manmade water based supernatant of fresh or dried Ephedra alata plant parts that comprises active ingredients, agents or compounds of Ephedra alata that are present in the supernatant at concentrated levels relative to their levels in the plant parts, wherein such active ingredients, agents or compounds are essentially free of Ephedra alata agents or compounds that are not water soluble. By essentially free of Ephedra alata agents or compounds that are not water soluble, it is predicted that the Ephedra alata aqueous extract is at least 80%, 85%, 90%, 95%, 98%, ₒᵣ 99% free of non-water soluble Ephedra alata agents or compounds The manmade water based supernatant of fresh or dried Ephedra alata plant parts is, therefore, a synthetic non-natural composition that can only be generated via human intervention.

It is, moreover, noteworthy that the leaves (approximately 5% of the Ephedra alata plant) comprise approximately 10-15% of the active ingredients (weight/weight %). In contrast, the active ingredients are present in extremely low amounts in the branches of the plant. Accordingly, the active ingredients, agents or compounds of Ephedra alata that are present in Ephedra alata powder or Ephedra alata aqueous extract made in accordance with methods presented herein are present at concentrated levels relative to their levels in the leaves or leaves and small branches from which they are generated and purified.

For intravenous injection, or injection at the site of affliction, the active ingredient may be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilizers, buffers, antioxidants and/or other additives may be included, as required.

A composition may be administered alone or in combination with other treatments, therapeutics or agents, either simultaneously or sequentially, dependent upon the condition to be treated. In addition, the present invention contemplates and includes compositions comprising the extract, agents and compounds herein described and other agents or therapeutics such as immune modulators, antibodies, immune cell stimulators, or adjuvants. In addition, the composition may be administered with hormones, such as dexamethasone, immune modulators, such as interleukins, tumor necrosis factor (TNF) or other growth factors, colony stimulating factors, or cytokines which stimulate the immune response. The composition may also be administered with, or may include combinations along with immune cell antigen antibodies or immune cell modulators.

The preparation of therapeutic compositions which contain agents or compounds as active ingredients is well understood in the art. Typically, such compositions are prepared as injectables, either as liquid solutions or suspensions. However, solid forms suitable for solution in, or suspension in, liquid prior to injection can also be prepared. The preparation can also be emulsified. The active therapeutic ingredient is often mixed with excipients which are pharmaceutically acceptable and compatible with the active ingredient. Suitable excipients are, for example, water, saline, dextrose, glycerol, ethanol, or the like and combinations thereof. In addition, if desired, the composition can contain minor amounts of auxiliary substances such as wetting or emulsifying agents, pH buffering agents which enhance the effectiveness of the active ingredient.

Agents or compounds can be formulated into a therapeutic composition as neutralized pharmaceutically acceptable salt forms. Pharmaceutically acceptable salts include the acid addition salts (formed with the free amino groups of the polypeptide or antibody molecule) and which are formed with inorganic acids such as, for example, hydrochloric or phosphoric acids, or such organic acids as acetic, oxalic, tartaric, mandelic, and the like. Salts formed from the free carboxyl groups can also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

The agent or compound containing compositions are conventionally administered intramuscularly, intravenously, as by injection of a unit dose, or orally, for example. The term “unit dose” when used in reference to a therapeutic composition of the present invention refers to physically discrete units suitable as unitary dosage for humans, each unit containing a predetermined quantity of active material calculated to produce the desired therapeutic effect in association with the required diluent; i.e., carrier, or vehicle.

The compositions are administered in a manner compatible with the dosage formulation, and in a therapeutically effective amount. The quantity to be administered depends on the subject to be treated, capacity of the subject's immune system to utilize the active ingredient, and degree of activation and immune response desired. Precise amounts of active ingredient required to be administered depend on the judgment of the practitioner and are peculiar to each individual. Suitable regimens for initial administration and follow on administration are also variable, and may include an initial administration followed by repeated doses at appropriate intervals by a subsequent injection or other administration.

The invention may be better understood by reference to the following non-limiting Examples, which are provided as exemplary of the invention. The following examples are presented in order to more fully illustrate the preferred embodiments of the invention and should in no way be construed, however, as limiting the broad scope of the invention.

### EXAMPLE 1

### Experimental Details

Method of Extraction:

This Example describes the preparation of extracts of Ephedra alata. The processes described below can be scaled up to produce larger quantities of extracts. The details provided for preparation of the following extracts represent a particular embodiment of the present methods for extract preparation and should not be considered as limiting. The quantities and times described below can be varied substantially to provide suitable extracts in accordance with the invention.

Accordingly, in a particular embodiment, 140 g of fresh or dried Ephedra alata plant is boiled in 6 L of water. The supernatant can be used directly for use on an animal or a human or dried under lyophilization conditions. The dried material can be re-constituted in aqueous solution or other formulation for the purpose of in vitro and in vivo testing.

Extract concentration: After lyophilization, the concentration of the crude extract is 0.5 mg/ml.

Dosing/Treatments:

Patients treated in accordance with methods presented herein are described in Tables 1 and 2. All patients received the aqueous extract (0.5 mg/ml). Each patient received twice daily 50 ml (equal to 25 mg in each dose) of the aqueous extract administered orally. The length of the treatments was dependent on the kind of disease as well as the disease state (early or late state) with which the patient presented. In general the treatment length ranged from 1-24 months (Table 3).

As shown in Tables 1 and 2, patients afflicted by a variety of inflammatory and autoimmune diseases and neurodegenerative diseases exhibited significant improvement as assessed by a reduction in the number of and severity of clinical symptoms and as visualized using various scanning/detection techniques.

Analytical Examination

The quality of the extract can be controlled by HPLC to monitor the concentration of each ingredient and accordingly the dose can be optimized.

### EXAMPLE 2

Progressive/Ataxia EAE Model

By way of background, various animal models of multiple sclerosis (MS) have been described. In the HLA-DR4 transgenic (tg) mice mouse model, for example, the mice develop atypical EAE. This strain of mouse was later demonstrated to exhibit ataxia as well. The HLA-DR2 tg mouse strain also develops EAE and is considered an animal model of MS. For the present experiments, HLA-DR2 tg mice were backcrossed to the TNFR2 KO mice to generate a progressive/ataxia EAE mouse model. See, for example, Forsthuber et al. (2001, J Immunol 167:7119-7125), Kawamura et al. (2008, J Immunol 181:3202-3211), and the editorial synopsis (2013, J Immunol 191:4891-4892), the entire content of each of which is incorporated herein by reference, for additional details.

Methods:

Progressive/ataxia Experimental Autoimmune Encephalomyelitis (EAE) model (experiments were performed in collaboration with the Forsthuber lab, San Antonio, Tex.): EAE in HLA-DR2 transgenic and TNF receptor 2 (TNFR2) knockout mice: the mice develop EAE which is characterized by strong MS-like ataxia. Results using these mice produce an “EAE” score, and an “ataxia” score.

10 Female and 10 Male DR15+/+la−/− TNFR2−/− mice were immunized with 200 μg MOG:CFA and 400 ng PTX (Day 0 and day 2) to induce disease. Mice were treated with 100 μg ephera [Ephedra alata extract (EFI)] in 200 μl nH₂O (buffered H₂O) or mock (equal volume nH₂O) starting at day 7. As a reference, the control group comprising 2 Females had a disease score of 5 on day 17.

Treatment was stopped at day 20 and mice were monitored for an additional 18 days. The reduction in the clinical score and ataxia scores was preserved over this period. Mice were sacrificed according to the animal protocol on day 27.

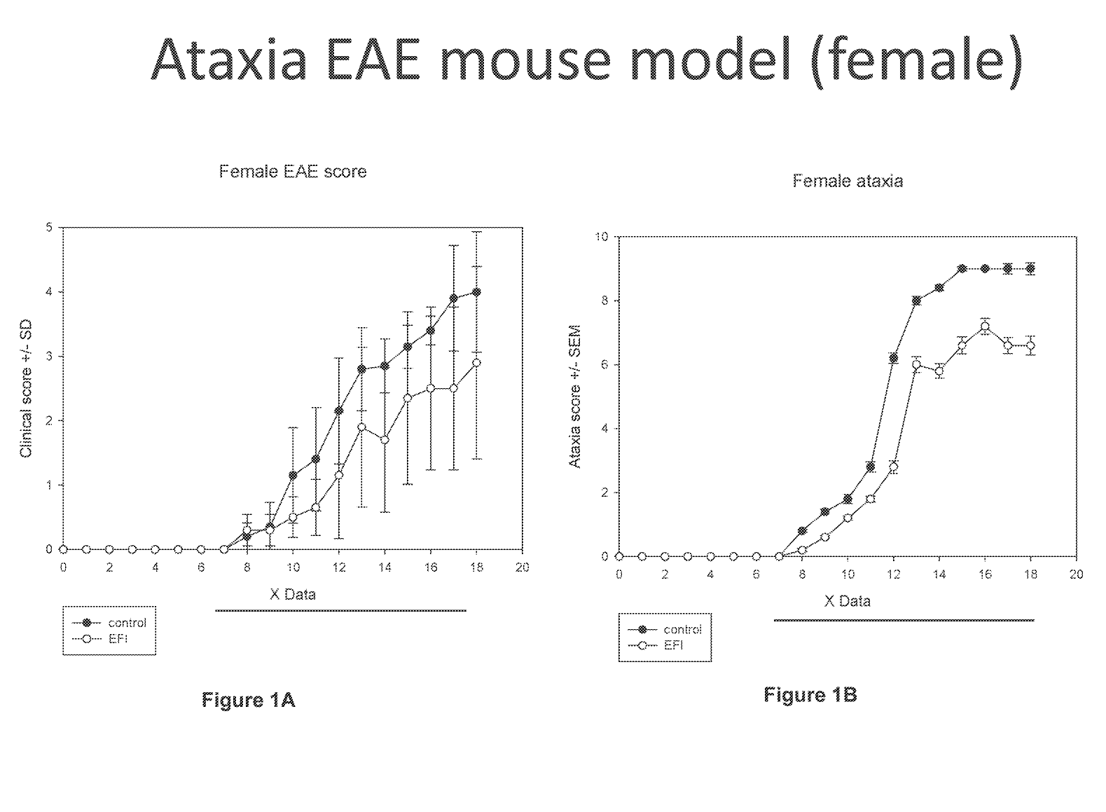

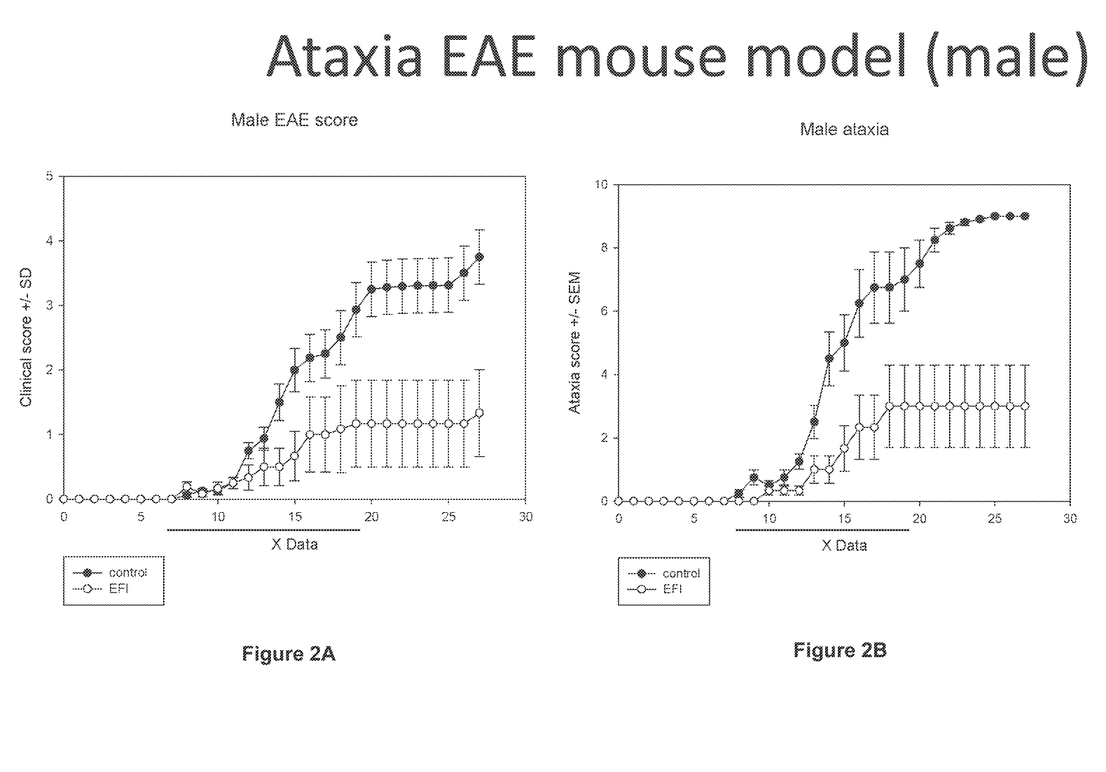

As shown in FIGS. 1 and 2, administering EFI to the progressive/ataxia EAE mice conferred symptomatic relief to the animals as reflected in improved EAE scores and ataxa scores. The ataxia score is significantly improved in both female and male mice treated with EFI. The improved EAE score is particularly pronounced in male mice treated with EFI.

While certain of the particular embodiments of the present invention have been described and specifically exemplified above, it is not intended that the invention be limited to such embodiments. Various modifications may be made thereto without departing from the scope and spirit of the present invention, as set forth in the following claims.

What is claimed is:

1. An aqueous extract or powder or a constituent thereof generated from Ephedra alata.

2. The aqueous extract or powder or constituent thereof of claim 1, wherein the aqueous extract or powder or constituent thereof comprises at least one of p-coumaric, ephedralone or derivatives thereof, furanofuran lignan, syringaresinol, nilocitin and digallooylglucose.

3. The aqueous extract or powder or constituent thereof of claim 1, wherein the aqueous extract or powder or constituent thereof comprises at least one of alkaloidal, lignan and phenolic constituents of Ephedra alata.

4. A composition comprising the aqueous extract or powder or constituent thereof of claim 1 and a pharmaceutically acceptable excipient or carrier.

5. A method for making the aqueous extract of claim 1, the method comprising:
processing an Ephedra alata plant or specific parts thereof in an aqueous solution to generate a supernatant comprising solubilized agents and insoluble material; and
removing the insoluble material from the supernatant, thereby generating the Ephedra alata aqueous extract.

6. The method of claim 5, wherein the processing comprises boiling fresh or dried Ephedra alata plant or specific parts thereof in water.

7. The method according to any one of claims 5-6, wherein the removing comprises gravity separation, centrifugation, and/or sieving through a filter.

8. The method according to any one of claims 5-7, wherein the Ephedra alata plant or specific parts thereof are fresh or dehydrated.

9. The method according to any one of claims 5-8, wherein the specific parts are leaves and small branches or leaves of the Ephedra alata plant.

10. The method according to any one of claims 5-9, further comprising reducing volume of the Ephedra alata aqueous extract to generate a concentrated Ephedra alata aqueous extract.

11. The method of claim 10, wherein the reducing is achieved by lyophilization.

12. The method according to any one of claims 5-11, further comprising mixing the Ephedra alata aqueous extract or the concentrated Ephedra alata aqueous extract with a pharmaceutically acceptable excipient or carrier.

13. A method for making the powder of claim 1, the method comprising: processing an Ephedra alata plant or specific parts thereof to generate a dehydrated Ephedra alata plant product; and grinding the dehydrated Ephedra alata plant product to generate a ground mixture thereof, thereby generating the Ephedra alata powder.

14. The method of claim 13, wherein the processing comprises washing and drying the Ephedra alata plant or specific parts thereof.

15. The method of claim 14, wherein the processing further comprises removal of large branches.

16. The method according to any one of claims 12-15, wherein the specific parts are leaves and small branches or leaves of the Ephedra alata plant.

17. The method according to any one of claims 12-16, further comprising sieving and regrinding the ground mixture.

18. The method according to any one of claims 12-17, further comprising mixing the Ephedra alata powder with a pharmaceutically acceptable excipient or carrier.

19. A method for treating a subject in need thereof with the Ephedra alata aqueous extract or powder or a constituent thereof according to any one of claims 1-3 or the composition according to claim 4, the method comprising administering a therapeutically effective amount of the Ephedra alata aqueous extract or powder or a constituent thereof or the composition to the subject in need thereof.

20. The method according to claim 19, wherein the subject is a mammal.

21. The method of claim 13, wherein the mammal is a human.

22. The method according to any one of claims 19-21, wherein the subject is afflicted with an inflammatory disease, an autoimmune disease, a cancer, a viral disease, or a neurodegenerative disease.

23. The method according to any one of claims 19-22, wherein the subject is afflicted with a condition or disease listed in Table 1 or Table 2.

24. The method according to any one of claims 19-23, wherein the subject is afflicted with an inflammatory disease or an autoimmune disease.

25. The method according to any one of claims 19-24, wherein the subject is afflicted with multiple sclerosis or psoriasis.

26. The method of according to any one of claims 19-25, wherein the Ephedra alata aqueous extract or powder or a constituent thereof or the composition is administered orally, topically, intratumorally, intranasally, intramuscularly, or intravenously.

27. The method of according to claim 26, wherein the Ephedra alata aqueous extract or powder or a constituent thereof or the composition is administered orally.

28. A method for formulating the Ephedra alata aqueous extract or powder or a constituent thereof according to any one of claims 1-3 or the composition according to claim 4 into a capsule, a tablet, a topical lotion, or a nasal spray, or a solution suitable for intravenous administration.

29. Ephedra alata aqueous extract or powder or a constituent thereof according to any one of claims 1-3 or the composition according to claim 4 for use in treating an inflammatory disease, an autoimmune disease, a cancer, a viral disease, or a neurodegenerative disease comprising administering the Ephedra alata aqueous extract or powder or a constituent thereof or the composition in a therapeutically effective amount to a subject afflicted with the inflammatory disease, autoimmune disease, cancer, viral disease, or neurodegenerative disease.

30. The use according to claim 29, wherein the disease is an inflammatory disease or an autoimmune disease.

31. The use according to any one of claims 29-30, wherein the disease is multiple sclerosis or psoriasis.

32. Use of Ephedra alata aqueous extract or powder or a constituent thereof according to any one of claims 1-3 or the composition according to claim 4 in the preparation of a medicament for treatment of an inflammatory disease, an autoimmune disease, a cancer, a viral disease, or a neurodegenerative disease.

33. The medicament according to claim 32, wherein the disease is an inflammatory disease or an autoimmune disease.

34. The medicament according to any one of claims 32-33, wherein the disease is multiple sclerosis or psoriasis.
